(19) United States
(12) Patent Application Publication
(10) Pub. No.: US 2022/0151908 A1
(43) Pub. Date: May 19, 2022

(54) POLYMER OIL BLEND

(71) Applicants: Dow Global Technologies LLC, Midland, MI (US); Rohm and Haas Company, Collegeville, PA (US)

(72) Inventors: Luqing Qi, Midland, MI (US); Liang Chen, Sewickley, PA (US); Lu Bai, Novi, MI (US); Lyndsay M. Leal, Spring City, PA (US); David M. Meunier, Midland, MI (US); Yunshen Chen, Lexington, MA (US); Fanwen Zeng, Audubon, PA (US)

(21) Appl. No.: 17/440,402

(22) PCT Filed: Apr. 16, 2020
(86) PCT No.: PCT/US2020/028423

§ 371 Date: Sep. 17, 2021

Related U.S. Application Data
(60) Provisional application No. 62/840,484, filed on Apr. 30, 2019.

(51) Int. Cl. A61K 8/81; A61K 8/37; A61K 8/92; A61Q 19/00; A61Q 5/00

(52) U.S. Cl. CPC A61K 8/8152; A61K 8/375; A61K 2800/48; A61Q 19/00; A61Q 5/00; A61K 8/922

(57) ABSTRACT

A polymer oil blend is provided comprising (a) a cosmetically acceptable long chain hydrophobic ester oil, wherein the cosmetically acceptable long chain hydrophobic ester oil comprises aliphatic C_{>12-24} alkyl triglycerides; (b) a thickening polymer powder, wherein the thickening polymer powder comprises: (i) 96 to 99.9 wt %, based on weight of the thickening polymer powder, of structural units of C₄₋₈ alkyl (meth)acrylate monomer; (ii) 0.1 to 2 wt %, based on weight of the thickening polymer powder, of structural units of (meth)acrylic acid monomer; (iii) 0 to 2 wt %, based on weight of the thickening polymer powder, of structural units of multi-ethylenically unsaturated crosslinking monomer; and (c) a silica powder; and wherein the weight ratio of silica powder/thickening polymer powder is ≥0.25. Also provided are personal care compositions containing same and methods of using same.

The present invention relates to a polymer oil blend for use in personal care applications. In particular, the present invention relates to a polymer oil blend comprising: (a) a cosmetically acceptable long chain hydrophobic ester oil, wherein the cosmetically acceptable long chain hydrophobic ester oil comprises aliphatic C_{>12-24} alkyl triglycerides; (b) a thickening polymer powder, wherein the thickening polymer powder comprises: (i) 96 to 99.9 wt %, based on weight of the thickening polymer powder, of structural units of C₄₋₈ alkyl (meth)acrylate monomer; (ii) 0.1 to 2 wt %, based on weight of the thickening polymer powder, of structural units of (meth)acrylic acid monomer; (iii) 0 to 2 wt %, based on weight of the thickening polymer powder, of structural units of multi-ethylenically unsaturated crosslinking monomer; and (c) a silica powder; and wherein the weight ratio of silica powder/thickening polymer powder is ≥0.25.

Personal care compositions include a variety of additives to provide an array of benefits to the composition. One such class of additives are oil thickeners that provide viscosity enhancements and impart good aesthetics, such as good sensory feel and clarity. Oil thickening agents that are known in the art include, for example, styrene-ethylene/butadiene-styrene copolymers, polyamide polymers and cellulose based polymers. These thickeners, however, come with certain drawbacks, including insufficient viscosity enhancement, high formulation temperature and lack of consistency in viscosity control in consumer product formulations.

An approach to the oil cleanser thickening compositions is disclosed in International Patent Application No. WO 2018/231953 to Bai, et al. Bai, et al. disclose a personal care composition comprising: (a) at least one cosmetically acceptable hydrophobic ester oil; (b) at least one surfactant selected from the group consisting of nonionic surfactants, anionic surfactants, and mixtures thereof; and (c) one or more polymers comprising polymerized structural units of (i) 79 to 95.74 weight % of C₄-C₈ (meth)acrylate monomers, (ii) 0.5 to 5 weight % of (meth)acrylic acid monomer, (iii) 3.75 to 14 weight % of (methoxy) poly(ethylene glycol) methacrylates, and (iv) 0.01 to 2 weight % of at least one crosslinker.

Notwithstanding, there remains a continuing need for an polymer oil blends for use in personal care formulations that impart desirable rheology and aesthetic characteristics to the incorporating personal care formulations.

The present invention provides a polymer oil blend comprising: (a) a cosmetically acceptable long chain hydrophobic ester oil, wherein the cosmetically acceptable long chain hydrophobic ester oil comprises aliphatic C_{>12-24} alkyl triglycerides; (b) a thickening polymer powder, wherein the thickening polymer powder comprises: (i) 96 to 99.9 wt %, based on weight of the thickening polymer powder, of structural units of C₄₋₈ alkyl (meth)acrylate monomer; (ii) 0.1 to 2 wt %, based on weight of the thickening polymer powder, of structural units of (meth)acrylic acid monomer; (iii) 0 to 2 wt %, based on weight of the thickening polymer powder, of structural units of multi-ethylenically unsaturated crosslinking monomer; and (c) a silica powder; and wherein the weight ratio of silica powder/thickening polymer powder is ≥0.25.

The present invention provides a personal care composition comprising 25 to 100 wt %, based on weight of the personal care composition, of a polymer oil blend of the present invention.

The present invention provides a method of treating skin or hair, comprising: providing a personal care composition of the present invention, and applying the personal care composition to at least one of skin and hair.

### DETAILED DESCRIPTION

We have now surprisingly found the unique polymer oil blend, as described herein, provide desired non-stringy aesthetic feel; effective thickening (preferably ≥100 Pa·s {more preferably, ≥200 Pa·s} measured at an angular frequency of 0.1 rad/s; shear thinning, wherein the viscosity measured under identical conditions at an angular frequency of 0.1 rad/s is ≥30% {preferably, ≥150%; more preferably, ≥300%} higher than that measured at an angular frequency of 100 rad/s). Examples of personal care compositions that may benefit from the polymer oil blend of the present invention as a sensory modifier include facial care, body care, hand cream, sunscreen, deodorant and cosmetic compositions.

Unless otherwise indicated, ratios, percentages, parts, and the like are by weight.

As used herein, unless otherwise indicated, the terms “weight average molecular weight” and “M_{w}” are used interchangeably to refer to the weight average molecular weight as measured in a conventional manner with gel permeation chromatography (GPC) and conventional standards, such as polystyrene standards. GPC techniques are discussed in detail in Modern Size Exclusion Liquid Chromatography: Practice of Gel Permeation and Gel Filtration Chromatography, Second Edition, Striegel, et al., John Wiley & Sons, 2009. Weight average molecular weights are reported herein in units of Daltons.

The term “polymer” as used herein and in the appended claims refers to a compound prepared by polymerizing monomers, whether of the same or a different type. The generic term “polymer” includes the terms “homopolymer,” “copolymer,” and “terpolymer.”

Percentages of monomer units in a polymer are percentages of solids or neat monomer weight, i.e., excluding any water present in a polymer emulsion.

The term “cosmetically acceptable” as used herein and in the appended refers to ingredients typically used in personal care compositions, and is intended to underscore that materials that are toxic when present in the amounts typically found in personal care compositions are not contemplated as part of the present invention.

The term “structural units” as used herein and in the appended claims refers to the remnant of the indicated monomer; thus a structural unit of 2-ethylhexyl acrylate is illustrated:

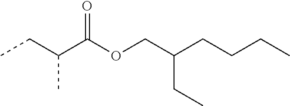

where the dotted lines represent the points of attachment to the polymer backbone.

The term “aesthetic characteristics” as used herein and in the appended claims in reference to an acidic aqueous cleansing formulation refers to visual and tactile sensory properties (e.g., smoothness, tack, lubricity, texture, color, clarity, turbidity, uniformity).

Preferably, the polymer oil blend of the present invention, comprises: (a) (preferably, 85 to 99.5 wt % (more preferably, 90 to 98 wt %; still more preferably, 92 to 96 wt %; most preferably, 93 to 95 wt %), based on weight of the polymer oil blend, of) a cosmetically acceptable long chain hydrophobic ester oil, wherein the cosmetically acceptable long chain hydrophobic ester oil comprises aliphatic C_{>12-24} alkyl triglycerides; (b) (preferably, 0.10 to 14.9 wt % (more preferably, 0.4 to 9.6 wt %; still more preferably, 0.75 to 7.25 wt %; most preferably, 1 to 6 wt %), based on weight of the polymer oil blend, of) a thickening polymer powder, wherein the thickening polymer powder comprises: (i) 96 to 99.9 wt % (preferably, 97 to 99.8 wt %; more preferably, 98 to 99.7 wt %; most preferably, 98.5 to 99.5 wt %), based on weight of the thickening polymer powder, of structural units of C₄₋₈ alkyl (meth)acrylate monomer; (ii) 0.1 to 4 wt % (preferably, 0.2 to 3 wt %; more preferably, 0.3 to 2 wt %; most preferably, 0.5 to 1.5 wt %), based on weight of the thickening polymer powder, of structural units of (meth)acrylic acid monomer; (iii) 0 to 2 wt % (preferably, 0.01 to 1 wt %; more preferably, 0.05 to 0.5 wt %; most preferably, 0.075 to 0.35 wt %), based on weight of the thickening polymer powder, of structural units of multi-ethylenically unsaturated crosslinking monomer; and (c) (preferably, 0.10 to 14.9 wt % (more preferably, 0.4 to 9.6 wt %; still more preferably, 0.75 to 7.25 wt %; most preferably, 1 to 6 wt %), based on weight of the polymer oil blend, of) a silica powder; and wherein the weight ratio of silica powder/thickening polymer powder is ≥0.25 (preferably, 0.25 to 2.0; more preferably, 0.3 to 1.75; still more preferably, 0.325 to 1.5; yet more preferably, 0.35 to 1.0; yet still more preferably, 0.375 to 0.75; most preferably, 0.38 to 0.5).

Preferably, the polymer oil blend of the present invention, comprises: a cosmetically acceptable long chain hydrophobic ester oil, wherein the cosmetically acceptable long chain hydrophobic ester oil comprises aliphatic C_{>12-24} alkyl triglycerides. More preferably, the polymer oil blend of the present invention, comprises: 85 to 99.5 wt % (preferably, 90 to 98 wt %; more preferably, 92 to 96 wt %; most preferably, 93 to 95 wt %), based on weight of the polymer oil blend, of a cosmetically acceptable long chain hydrophobic ester oil; wherein the cosmetically acceptable long chain hydrophobic ester oil comprises aliphatic C_{>12-24} alkyl triglycerides. Most preferably, the polymer oil blend of the present invention, comprises: 85 to 99.5 wt % (preferably, 90 to 98 wt %; more preferably, 92 to 96 wt %; most preferably, 93 to 95 wt %), based on weight of the polymer oil blend, of a cosmetically acceptable long chain hydrophobic ester oil; wherein the cosmetically acceptable long chain hydrophobic ester oil comprises aliphatic C_{>12-24} alkyl triglycerides; wherein the polymer oil blend comprises <1 wt % (preferably, <0.1 wt %; more preferably, <0.01 wt %; most preferably, <detectable limit), based on weight of the polymer oil blend, of hydrophobic ester oil of C₈₋₁₂ alkyl triglycerides.

Preferably, the polymer oil blend of the present invention, comprises: a cosmetically acceptable long chain hydrophobic ester oil, wherein the cosmetically acceptable long chain hydrophobic ester oil comprises aliphatic C_{>12-24} alkyl triglycerides; and wherein the cosmetically acceptable long chain hydrophobic ester oil is selected from the group consisting of almond oil, andiroba oil, apricot kernel oil, argan oil, avacado oil, babassu oil, borage oil, canola oil, castor oil, coca butter, coconut oil, corn oil, cottonseed oil, crambe oil, cupuacu butter, evening primrose, grape seed oil, hazelnut oil, hybrid safflower oil, illipe butter, Japan wax, jatropha oil, jojoba oil, kokhum butter, linseed oil, mango butter, meadowfoam oil, milk fat, olive oil, ongokea oil, palm kernel oil, palm oil, peanut oil, poppyseed oil, rapeseed oil, rice bran oil, safflower oil, sesame oil, shea butter, soybean oil, sunflower oil, sweet almond oil, tallow, tung oil, walnut oil, wheat germ oil, veronia oil and mixtures thereof. More preferably, the polymer oil blend of the present invention, comprises: 85 to 99.5 wt % (preferably, 90 to 98 wt %; more preferably, 92 to 96 wt %; most preferably, 93 to 95 wt %), based on weight of the polymer oil blend, of a cosmetically acceptable long chain hydrophobic ester oil; wherein the cosmetically acceptable long chain hydrophobic ester oil comprises aliphatic C_{>}12-24 alkyl triglycerides; and wherein the cosmetically acceptable long chain hydrophobic ester oil is selected from the group consisting of almond oil, andiroba oil, apricot kernel oil, argan oil, avacado oil, babassu oil, borage oil, canola oil, castor oil, coca butter, coconut oil, corn oil, cottonseed oil, crambe oil, cupuacu butter, evening primrose, grape seed oil, hazelnut oil, hybrid safflower oil, illipe butter, Japan wax, jatropha oil, jojoba oil, kokhum butter, linseed oil, mango butter, meadowfoam oil, milk fat, olive oil, ongokea oil, palm kernel oil, palm oil, peanut oil, poppyseed oil, rapeseed oil, rice bran oil, safflower oil, sesame oil, shea butter, soybean oil, sunflower oil, sweet almond oil, tallow, tung oil, walnut oil, wheat germ oil, veronia oil and mixtures thereof. Most preferably, the polymer oil blend of the present invention, comprises: 85 to 99.5 wt % (preferably, 90 to 98 wt %; more preferably, 92 to 96 wt %; most preferably, 93 to 95 wt %), based on weight of the polymer oil blend, of a cosmetically acceptable long chain hydrophobic ester oil; wherein the cosmetically acceptable long chain hydrophobic ester oil comprises aliphatic C_{>12-24} alkyl triglycerides; wherein the cosmetically acceptable long chain hydrophobic ester oil is selected from the group consisting of almond oil, argan oil, borage oil, canola oil, castor oil, corn oil, cottonseed oil, crambe oil, jatropha oil, jojoba oil, olive oil, palm oil, peanut oil, rapeseed oil, soybean oil, sunflower oil and mixtures thereof (preferably, corn oil, cotton seed oil, soybean oil, sunflower oil and mixtures thereof; more preferably, corn oil, sunflower oil and mixtures thereof; most preferably, sunflower oil); and wherein the polymer oil blend comprises <1 wt % (preferably, <0.1 wt %; more preferably, <0.01 wt %; most preferably, <detectable limit), based on weight of the polymer oil blend, of hydrophobic ester oil of C₈₋₁₂ alkyl triglycerides.

Preferably, the polymer oil blend of the present invention, comprises: a thickening polymer powder. More preferably, the polymer oil blend of the present invention, comprises: 0.10 to 14.9 wt % (preferably, 0.4 to 9.6 wt %; more preferably, 0.75 to 7.25 wt %; most preferably, 1 to 6 wt %), based on weight of the polymer oil blend, of a thickening polymer powder. Most preferably, the polymer oil blend of the present invention, comprises: 0.10 to 14.9 wt % (preferably, 0.4 to 9.6 wt %; more preferably, 0.75 to 7.25 wt %; most preferably, 1 to 6 wt %), based on weight of the polymer oil blend, of a thickening polymer powder; wherein the thickening polymer comprises: (i) 96 to 99.9 wt % (preferably, 97 to 99.8 wt %; more preferably, 98 to 99.7 wt %; most preferably, 98.5 to 99.5 wt %), based on weight of the thickening polymer powder, of structural units of C₄₋₈ alkyl (meth)acrylate monomer (preferably, at least one of 2-ethylhexyl (meth)acrylate, n-butyl (meth)acrylate, iso-butyl (meth)acrylate, t-butyl (meth)acrylate and mixtures thereof; more preferably, 2-ethylhexyl (meth)acrylate, n-butyl (meth)acrylate, iso-butyl (meth)acrylate and mixtures thereof; most preferably, 2-ethylhexyl (meth)acrylate and iso-butyl (meth)acrylate); (ii) 0.1 to 2 wt %, based on weight of the thickening polymer powder, of structural units of (meth)acrylic acid monomer (preferably, methacrylic acid); (iii) 0 to 2 wt %, based on weight of the thickening polymer powder, of structural units of multi-ethylenically unsaturated crosslinking monomer (preferably, at least one of trimethylolpropane trimethacrylate and trimethylolpropane diallyl ether; more preferably, trimethylolpropane trimethacrylate).

Preferably, the thickening polymer in emulsion, before drying into powder form, has an average particle size of 50 nm to 2 μm, as measured by a Brookhaven BI-90. More preferably, the thickening polymer in emulsion, before drying into powder form, has an average particle size of 75 nm to 1.1 μm, as measured by a Brookhaven BI-90. Most preferably, the thickening polymer in emulsion, before drying into powder form, has an average particle size of 100 to 250 nm, as measured by a Brookhaven BI-90.

Preferably, the thickening polymer powder comprises 96 to 99.9 wt % (preferably, 97 to 99.8 wt %; more preferably, 98 to 99.7 wt %; most preferably, 98.5 to 99.5 wt %), based on weight of the thickening polymer powder, of structural units of C₄₋₈ alkyl (meth)acrylate monomer. Preferably, the C₄₋₈ alkyl (meth)acrylate monomer is selected from the group consisting of at least one of 2-ethylhexyl (meth)acrylate, n-butyl (meth)acrylate, iso-butyl (meth)acrylate, t-butyl (meth)acrylate and mixtures thereof. More preferably, the C₄₋₈ alkyl (meth)acrylate monomer includes at least one of 2-ethylhexyl (meth)acrylate, n-butyl (meth)acrylate and iso-butyl (meth)acrylate. Most preferably, the C₄₋₈ alkyl (meth)acrylate monomer includes 2-ethylhexyl (meth)acrylate and iso-butyl (meth)acrylate).

Preferably, the thickening polymer powder comprises 96 to 99.9 wt % (preferably, 97 to 99.8 wt %; more preferably, 98 to 99.7 wt %; most preferably, 98.5 to 99.5 wt %), based on weight of the thickening polymer powder, of structural units of C₄₋₈ alkyl (meth)acrylate monomer; wherein 40 to 90 wt % (preferably, 45 to 85 wt %; more preferably, 45 to 55 wt %; still more preferably, 48 to 52 wt %; most preferably, 49 to 51 wt %) of the structural units of C₄₋₈ alkyl (meth)acrylate monomer are derived from a C₄ alkyl (meth)acrylate monomer; and wherein 10 to 60 wt % (preferably, 15 to 55 wt %; more preferably, 45 to 55 wt %; still more preferably, 48 to 52 wt %; most preferably, 49 to 51 wt %) of the structural units of C₄₋₈ alkyl (meth)acrylate monomer are derived from a C₈ alkyl (meth)acrylate monomer. More preferably, the thickening polymer powder comprises 96 to 99.9 wt % (preferably, 97 to 99.8 wt %; more preferably, 98 to 99.7 wt %; most preferably, 98.5 to 99.5 wt %), based on weight of the thickening polymer powder, of structural units of C₄₋₈ alkyl (meth)acrylate monomer; wherein 40 to 90 wt % (preferably, 45 to 85 wt %; more preferably, 45 to 55 wt %; still more preferably, 48 to 52 wt %; most preferably, 49 to 51 wt %) of the structural units of a C₄₋₈ alkyl (meth)acrylate monomer are derived from iso-butyl methacrylate; and wherein 10 to 60 wt % (preferably, 15 to 55 wt %; more preferably, 45 to 55 wt %; still more preferably, 48 to 52 wt %; most preferably, 49 to 51 wt %) of the structural units of C₄₋₈ alkyl (meth)acrylate monomer are derived from 2-ethylhexyl methacrylate.

Preferably, the thickening polymer powder comprises 0.1 to 4 wt % (preferably, 0.2 to 3 wt %; more preferably, 0.3 to 2 wt %; most preferably, 0.5 to 1.5 wt %), based on weight of the thickening polymer powder, of structural units of (meth)acrylic acid monomer. More preferably, the thickening polymer powder comprises 0.1 to 4 wt % (preferably, 0.2 to 3 wt %; more preferably, 0.3 to 2 wt %; most preferably, 0.5 to 1.5 wt %), based on weight of the thickening polymer powder, of structural units of methacrylic acid monomer.

Preferably, the thickening polymer powder comprises 0 to 2 wt %, based on weight of the thickening polymer powder, of structural units of a multi-ethylenically unsaturated crosslinking monomer. More preferably, the thickening polymer powder comprises 0.01 to 1 wt %, based on weight of the thickening polymer powder, of structural units of a multi-ethylenically unsaturated crosslinking monomer. Still more preferably, the thickening polymer powder comprises 0.05 to 0.5 wt %, based on weight of the thickening polymer powder, of structural units of a multi-ethylenically unsaturated crosslinking monomer. Most preferably, the thickening polymer powder comprises 0.075 to 0.35 wt %, based on weight of the thickening polymer powder, of structural units of a multi-ethylenically unsaturated crosslinking monomer.

Preferably, the multi-ethylenically unsaturated crosslinking monomer is selected from crosslinker monomers having two or more non-conjugated ethylenically unsaturated groups. More preferably, the multi-ethylenically unsaturated crosslinking monomer is selected from the group consisting of di- or tri-allyl ethers and di- or tri-(meth)acrylyl esters of diols or polyols (e.g., trimethylolpropane diallyl ether (TMPDE), trimethylol propane trimethacrylate (TMPTMA), ethylene glycol dimethacrylate (EGDMA), diethylene glycol dimethacylate (DEGDMA), 1,6-hexanediol dimethacrylate, polyethylene glycol diacrylate, and polypropylene glycol dimethacrylate); di- or tri-allyl esters of di- or tri-acids (e.g., diallyl phthalate); allyl (meth)acrylate; divinyl sulfone; triallyl phosphate; divinylaromatics (e.g., divinylbenzene); and mixtures thereof. Still more preferably, the multi-ethylenically unsaturated crosslinking monomer is selected from the group consisting of trimethylolpropane trimethacrylate, ethylene glycol dimethacrylate and mixtures thereof. Still more preferably, the multi-ethylenically unsaturated crosslinking monomer includes trimethylolpropane trimethacrylate. Most preferably, the multi-ethylenically unsaturated crosslinking monomer is trimethylolpropane trimethacrylate.

Preferably, the thickening polymer powder contains <1 wt %, based on weight of the thickening polymer powder, of structural units of (methoxy) poly(ethylene glycol) monomer. As used herein and in the appended claims, the term “(methoxy) poly(ethylene glycol) (meth)acrylate” means methoxy poly(ethylene glycol) methacrylate, methoxy poly(ethylene glycol) acrylate, poly(ethylene glycol) methacrylate and poly(ethylene glycol) acrylate. More preferably, the thickening polymer powder contains <1 wt % (preferably, <0.1 wt %; more preferably, <0.001 wt %; most preferably, <detectable limit), based on weight of the thickening polymer powder, of structural units of methoxy poly(ethylene glycol) methacrylate, methoxy poly(ethylene glycol) acrylate, poly(ethylene glycol) methacrylate and poly(ethylene glycol) acrylate. Most preferably, the thickening polymer powder contains <1 wt % (preferably, <0.1 wt %; more preferably, <0.001 wt %; most preferably, <detectable limit), based on weight of the thickening polymer powder, of structural units of methoxy poly(ethylene glycol) methacrylate, methoxy poly(ethylene glycol) acrylate, poly(ethylene glycol) methacrylate and poly(ethylene glycol) acrylate, collectively.

Preferably, the thickening polymer powder contains <1 wt % (preferably, <0.1 wt %; more preferably, <0.001 wt %; most preferably, <detectable limit), based on weight of the thickening polymer powder, of structural units of lipophilically modified (meth)acrylate monomers having the following structure

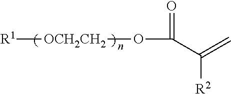

wherein R¹ is a linear saturated C₁₀₋₂₀ alkyl group (preferably, a linear saturated C₁₂₋₁₈ alkyl group; more preferably, a linear saturated C₁₂₋₁₄ alkyl group); R² is a hydrogen or a methyl group; and n is an average of 2 to 60 (preferably, 5 to 40; more preferably, 10 to 30).

Preferably, the thickening polymer powder is provided using at least one of spray drying, freeze drying and coagulation. More preferably, the thickening polymer powder is spray dried. In certain spray drying processes, anti-caking agents may be mixed with an acrylic polymer suspension prior to spray drying or introduced as a dry powder during the spray drying process. Anti-caking agents include mineral fillers (e.g., calcium carbonate, kaolin, titanium oxide, talc, hydrated alumina, bentonite and silica); solid polymer particles with a glass transition or melting temperature above 60° C. (e.g., polymethylmethacrylate, polystyrene and high density polyethylene); and water soluble polymers with a glass transition temperature above 60° C. (e.g., polyvinyl alcohol and methylcellulose). The anti-caking agents are used as flow aids to help prevent the dried polymer particles from sticking to each other or the processing equipment. Anti-caking agents may be included in the processing at up to 20 wt %, based on the weight of the collected polymer powder. Note that thickening polymer powder produced using silica as a flow aid surprisingly does not exhibit the same superior performance associated with physical blending of silica powder and thickening polymer powder in the invention as described and claimed herein.

Preferably, the polymer oil blend of the present invention, comprises: a silica powder. More preferably, the polymer oil blend of the present invention, comprises: 0.10 to 14.9 wt % (preferably, 0.4 to 9.6 wt %; more preferably, 0.75 to 7.25 wt %; most preferably, 1 to 6 wt %), based on weight of the polymer oil blend, of a silica powder. Still more preferably, the polymer oil blend of the present invention, comprises: 0.10 to 14.9 wt % (preferably, 0.4 to 9.6 wt %; more preferably, 0.75 to 7.25 wt %; most preferably, 1 to 6 wt %), based on weight of the polymer oil blend, of a silica powder; wherein the silica powder is a hydrophilic silica powder (preferably, wherein the silica powder is a fumed hydrophilic silica powder; more preferably, wherein the silica powder is an amorphous fumed hydrophilic silica powder; most preferably, wherein the silica powder is an amorphous, colloidal fumed hydrophilic silica powder). Most preferably, the polymer oil blend of the present invention, comprises: 0.10 to 14.9 wt % (preferably, 0.4 to 9.6 wt %; more preferably, 0.75 to 7.25 wt %; most preferably, 1 to 6 wt %), based on weight of the polymer oil blend, of a silica powder; wherein the silica powder is a hydrophilic silica powder (preferably, wherein the silica powder is a fumed hydrophilic silica powder; more preferably, wherein the silica powder is an amorphous fumed hydrophilic silica powder; most preferably, wherein the silica powder is an amorphous, colloidal fumed hydrophilic silica powder); and wherein the silica powder has an average aggregate particle length of 200 to 300 nanometers.

In preparing the polymer oil blend of the present invention, the thickening polymer powder and the silica powder are mixed as dry solids (i.e., wherein the thickening polymer is isolated in dry powder form before it is combined with the silica powder to form the polymer oil blend).

The polymer oil blend of the present invention is useful as a sensory agent or sensory modifier used to impart superior aesthetic feel to personal care compositions. In particular, the polymer oil blend of the present invention provides the desired non-stringy aesthetic feel; effective thickening (preferably ≥100 Pa·s {more preferably, ≥200 Pa·s} measured with a shear rate of 0.1 reciprocal second, s⁻; shear thinning, wherein the viscosity measured under identical conditions with a shear rate of 0.1 s⁻ is ≥30% {preferably, ≥150%; more preferably, ≥300%} higher than that measured with a shear rate of 100 s⁻). Examples of personal care compositions that may benefit from sensory modifiers include facial care, body care, hand cream, sunscreen, deodorant and cosmetic compositions.

Preferably, the personal care composition of the present invention, comprises: a polymer oil blend of the present invention. More preferably, the personal care composition of the present invention, comprises: 1 to 100 wt % (preferably, 40 to 95 wt %; more preferably, 45 to 80 wt %; most preferably, 50 to 75 wt %), based on weight of the personal care composition, of a polymer oil blend of the present invention. Most preferably, the personal care composition of the present invention, comprises: 25 to 100 wt % (preferably, 40 to 95 wt %; more preferably, 45 to 80 wt %; most preferably, 50 to 75 wt %), based on weight of the personal care composition, of a polymer oil blend of the present invention.

Preferably, the personal care composition of the present invention, optionally further comprises a vehicle. More preferably, the personal care composition of the present invention, optionally further comprises: a vehicle, wherein the vehicle is selected from the group consisting of water and an aqueous C₁₋₄ alcohol mixture. Most preferably, the personal care composition of the present invention, optionally further comprises: a vehicle, wherein the vehicle is water.

Preferably, the personal care composition of the present invention, comprises: 0 to 99 wt % (preferably, 5 to 60 wt %; more preferably, 20 to 55 wt %; most preferably, 25 to 50 wt %), based on weight of the personal care composition, of a vehicle. More preferably, the personal care composition of the present invention, comprises: 0 to 99 wt % (preferably, 5 to 60 wt %; more preferably, 20 to 55 wt %; most preferably, 25 to 50 wt %), based on weight of the personal care composition, of a vehicle; wherein the vehicle is selected from the group consisting of water and an aqueous C₁₋₄ alcohol mixture. Most preferably, the personal care composition of the present invention, comprises: 0 to 99 wt % (preferably, 5 to 60 wt %; more preferably, 20 to 55 wt %; most preferably, 25 to 50 wt %), based on weight of the personal care composition, of a vehicle; wherein the vehicle comprises water.

Preferably, the water used in the personal care composition of the present invention is at least one of distilled water and deionized water. More preferably, the water used in the personal care composition of the present invention is distilled and deionized.

Preferably, the personal care composition of the present invention may optionally further comprise at least one personal care additive selected from the group consisting of abrasives; absorbents; fragrances; pigments; colorings/colorants; essential oils; skin sensates; astringents (e.g., clove oil, menthol, camphor, eucalyptus oil, eugenol, menthyl lactate, witch hazel distillate); preservatives; anti-caking agents; foam builders; antifoaming agents; antimicrobial agents (e.g., iodopropyl butylcarbamate); antioxidants; binders; biological additives; buffering agents; bulking agents; chelating agents; chemical additives; cosmetic astringents; cosmetic biocides; denaturants; drug astringents; topical analgesics; film formers; opacifying agents; pH adjusters; propellants; reducing agents; sequestrants; skin bleaching and lightening agents (e.g., hydroquinone, kojic acid, ascorbic acid, magnesium ascorbyl phosphate, ascorbyl glucosamine); skin conditioning agents (e.g., hymectants); skin soothing agents (e.g., panthenol, aloe vera, pantothenic acid, allantoin, bisabolol, dipotassium glycyrrhizinate); skin treating agents; vitamins (e.g., Vitamin C); silicones and fatty alcohols.

Preferably, the personal care composition of the present invention further comprises a cosmetically acceptable surfactant. More preferably, the personal care composition of the present invention further comprises a cosmetically acceptable surfactant selected from the group consisting of anionic surfactants, nonionic surfactants, zwitterionic surfactants and mixtures thereof. Most preferably, the personal care composition of the present invention further comprises a cosmetically acceptable surfactant selected from the group consisting of anionic surfactants, nonionic surfactants and mixtures thereof.

Preferably, the personal care composition of the present invention, comprises 0 to 50 wt % (preferably, 1 to 50 wt %; more preferably, 5 to 30 wt %; most preferably, 10 to 25 wt %), based on weight of the personal care composition, of a cosmetically acceptable surfactant. More preferably, the personal care composition of the present invention comprises 0 to 50 wt % (preferably, 1 to 50 wt %; more preferably, 5 to 30 wt %; most preferably, 10 to 25 wt %), based on weight of the personal care composition, of a cosmetically acceptable surfactant selected from the group consisting of anionic surfactants, nonionic surfactants, zwitterionic surfactants and mixtures thereof. Most preferably, the personal care composition of the present invention comprises 0 to 50 wt % (preferably, 1 to 50 wt %; more preferably, 5 to 30 wt %; most preferably, 10 to 25 wt %), based on weight of the personal care composition, of a cosmetically acceptable surfactant selected from the group consisting of anionic surfactants, nonionic surfactants and mixtures thereof.

Preferably, the anionic surfactants used in the personal care composition of the present invention are selected from the group of cosmetically acceptable anionic surfactants. Preferably, the cosmetically acceptable anionic surfactants are selected from the group consisting of alkyl sulfates; alkyl ether sulfates; alkyl-substituted aryl sulfonates; alkyl succinates; alkyl sulfosuccinates; alkyl sarcosinates; α-olefin sulfonates; sodium, magnesium, ammonium, ethanolamine, diethanolamine and triethanolamine salts thereof; and mixtures thereof. More preferably, the cosmetically acceptable anionic surfactants are selected from the group consisting of C₈₋₁₈ alkyl sulfates; C₈₋₁₈ alkyl (EO)ₙ(PO)ₘ, sulfates, where n and m are independently 0 to 10 and where n+m is 1 to 10 (preferably, 2 to 3); C₈₋₁₈ alkyl-substituted aryl sulfonates; C₈₋₁₈ alkyl succinates; C₈₋₁₈ alkyl sulfosuccinates; C₈₋₁₈ alkyl sarcosinates; α-olefin sulfonates; sodium, magnesium, ammonium, ethanolamine, diethanolamine and triethanolamine salts thereof; and mixtures thereof. Still more preferably, the cosmetically acceptable anionic surfactants are selected from the group consisting of sodium lauryl sulfate, sodium octadecyl succinate, ammonium lauryl sulphosuccinate, ammonium lauryl sulfate, ammonium lauryl ether sulfate, sodium dodecylbenzene sulfonate, triethanolamine dodecylbenzene sulfonate, sodium N-lauryl sarcosinate and mixtures thereof. Most preferably, the cosmetically acceptable anionic surfactants are selected from the group consisting of, sodium lauryl sulfate, sodium lauryl (EO)₂ sulfate, sodium lauryl (EO)₃ sulfate, ammonium lauryl sulfate, ammonium lauryl (EO) sulfate, ammonium lauryl (EO)₂ sulfate, ammonium (EO)₃ sulfate, triethanolamine dodecylbenzene sulfonate and mixtures thereof.

Preferably, the nonionic surfactants used in the personal care composition of the present invention are selected from the group of cosmetically acceptable nonionic surfactants. Preferably, the cosmetically acceptable nonionic surfactants are selected from the group consisting of polyoxyalkylene surfactants, polyalkylene glycol esters, polyoxyethylene derivatives of fatty acid esters of polyhydric alcohols, fatty acid esters of polyalkoxylated polyhydric alcohols, polyalkoxylated natural fats and oils, polyalkylene oxide block copolymers, alkyl polyglucosides, sucrose esters and mixtures thereof. More preferably, the cosmetically acceptable nonionic surfactants are selected from polyoxyalkylene surfactants. Most preferably, the cosmetically acceptable nonionic surfactants are selected from polyoxyethylene surfactants. Preferred polyoxyethylene surfactants are selected from the group consisting of alcohol alkoxylates, alkylphenol alkoxylates and mixtures thereof. Preferred alcohol alkoxylates include, for example, alcohol ethoxylates and alcohol propoxylates. More preferred cosmetically acceptable nonionic surfactants include nonionic surfactants selected from the group consisting of alcohol ethoxylate that conforms to the formula

R²—(OCH₂CH₂)_{w}OH

wherein R² is a C₁₀₋₃₀ alkyl group (preferably, a C₁₂₋₂₆ alkyl group; more preferably, a C₁₂₋₂₀ alkyl group; most preferably, a C₁₂₋₁₈ alkyl group); and w has an average value of 10 to 200 (preferably, 10 to 160; more preferably, 12 to 140; most preferably, 20 to 100). Still more preferred cosmetically acceptable nonionic surfactants include nonionic surfactants selected from the group consisting of a polyethylene glycol ether of lauryl alcohol that conforms to the formula

CH₃(CH₂)₁₀CH₂(OCH₂CH₂)ₓOH

wherein x has an average value of 10 to 30 (preferably, 12 to 26; more preferably, 15 to 25; most preferably, 23); a polyethylene glycol ether of cetyl alcohol that conforms to the formula

CH₃(CH₂)₁₄CH₂(OCH₂CH₂)_{y}OH

wherein y has an average value of 10 to 30 (preferably, 12 to 26; more preferably, 15 to 25; most preferably, 20); a polyethylene glycol ether of stearyl alcohol that conforms to the formula

CH₃(CH₂)₁₆CH₂(OCH₂CH₂)_{z}OH

wherein z has an average value of 10 to 160 (preferably, 60 to 140; more preferably, 80 to 120; most preferably, 100); and mixtures thereof.

Preferably, the zwitterionic surfactants used in the personal care composition of the present invention are selected from the group of cosmetically acceptable zwitterionic surfactants. Preferably, the cosmetically acceptable zwitterionic surfactants are selected from the group consisting of alkyl amine oxides, alkyl betaines, alkyl amido propyl betaines, alkyl alkanol amides, alkyl di-alkanol amides, alkyl sulfobetaines, alkyl glycinates, alkyl carboxy glycinates and mixtures thereof. More preferably, the cosmetically acceptable zwitterionic surfactants are selected from the group consisting of C₈₋₁₈ alkyl amine oxides, C₈₋₁₈ alkyl betaines, C₈₋₁₈ alkyl amido propyl betaines, C₈₋₁₈ alkyl alkanol amides, C₈₋₁₈ alkyl di-alkanol amides, C₈₋₁₈ alkyl sulfobetaines, C₈₋₁₈ alkyl glycinates, C₈₋₁₈ alkyl carboxy glycinates and mixtures thereof. Preferred cosmetically acceptable zwitterionic surfactants include lauryl amine oxide, cocamide monoethanolamine, cocamide diethanolamine, cocamidopropyl betaine, cocodimethyl sulfopropyl betaine and mixtures thereof.

Preferably, the method of treating skin or hair of the present invention, comprises: providing a personal care composition of the present invention and applying the personal care composition to at least one of a skin and hair. More preferably, the method of using a personal care composition of the present invention, further comprises: rinsing the personal care composition from the at least one of skin and hair with a rinse water.

Some embodiments of the present invention will now be described in detail in the following Examples.

### Example S1: Synthesis of Polymer 1

To a three liter round bottom flask equipped with a mechanical overhead stirrer, a heating mantle, a thermocouple, a condenser and inlets for the addition of monomer, initiator and nitrogen was charged deionized water (470 g) and sodium dodecylbenzene sulfonate (7.46 g; DS-4 Polystep A-16-22 from Stepan). The flask contents were then set to stir with a nitrogen flow and heated to 87-89° C. In a separate plastic lined vessel with overhead stirring was added sodium dodecylbenzene sulfonate (7 g), deionized water (181.65 g), isobutyl methacrylate (277.2 g), 2-ethylhexyl methacrylate (277.2 g), methacrylic acid (5.6 g) and trimethylolpropane trimethacrylate (1.568 g) and allowed to form a smooth, stable monomer emulsion. An initial catalyst charge of ammonium persulfate (0.28 g) in deionized water (12.71 g) was prepared and set aside. A buffer solution of ammonium bicarbonate (1.92 g) in deionized water (12.71 g) was prepared and set aside. A preform seed of 22.38 grams was removed from the stable monomer emulsion and put into a small beaker. A rinse of deionized water (16.8 g) was prepared. A co-feed catalyst charge of ammonium persulfate (0.28 g) in deionized water (49.22 g) was prepared and set aside.

When the contents of the flask was at temperature, the buffer solution and initial catalyst charge were added to the flask contents, followed by addition of the preform seed and rinse. The reaction was monitored for a small exotherm. After the exotherm, the temperature control was adjusted to 83-85° C. The monomer emulsion was then added to the flask, sub-surface, at a rate of 4.38 g/min. for 15 minutes after which the rate was increased to 8.77 g/min. for 75 minutes. While the monomer emulsion was added to the flask, the co-feed catalyst solution was simultaneously added at a rate of 0.55 g/min. Upon completion of the monomer emulsion and co-feed catalyst additions, deionized water (16.8 g) was added as a rinse. The contents of the flask were then held for 20 minutes at 83-85° C.

During the hold, a chase promoter of 3.77 grams of a 0.15% iron sulfate heptahydrate solution was prepared. A chase activator solution of isoascorbic acid (1.12 g) dissolved in deionized water (36.40 g) was prepared. A chase catalyst solution of 70% tert-butyl hydroperoxide (2.14 g) in deionized water (35.40 g) was prepared.

At 80° C., the chase promoter solution was added as a shot to the flask. The flask contents were then cooled to 70° C., while adding the chase activator and chase catalyst solutions separately by syringe over 60 minutes at a feed rate of 0.7 g/min. The flask contents were then held for 10 minutes, and then cooled to room temperature. When the flask contents reached room temperature, the emulsion product was filtered through a 100 mesh bag.

The filtered emulsion product was then spray dried using a two-fluid nozzle atomizer equipped on a Mobile Minor spray dryer (GEA Process Engineering Inc.). The spray drying was performed under an inert nitrogen atmosphere. Nitrogen was supplied to the atomizer at ambient temperature, 1 bar and a 6.0 kg/hour flow rate. The polymer emulsion was fed into the atomizer at 30 mL/min using a peristaltic pump (Masterflex L/S). Heated nitrogen was used to evaporate the water. The inlet temperature was set at 140° C., and the outlet temperature was equilibrated at 40-50° C. by fine tuning the emulsion feed rate. The resulting polymer powder was collected in a glass jar attached to the cyclone and subsequently vacuum dried at room temperature to remove residual moisture.

### Example S2: Synthesis of Polymer 2

The polymer of Polymer 2 was prepared using substantially the same synthesis as described above in Example S1 for Polymer 1 with appropriate changes in monomer and monomer amounts as recited in TABLE 1.

TABLE 1

| | Monomer (wt %) | | | X-linker (wt %) | |
|---|---|---|---|---|---|
| Sample | iBMA | EHMA | MAA | TMPTMA | TMPDE |
| Polymer 1 | 49.5 | 49.5 | 1 | 0.28 | — |
| Polymer 2 | 79.5 | 19.5 | 1 | — | 0.1 |

iBMA = isobuty methacrylate
EHMA = 2-ethylhexyl methacrylate
MAA = methacrylic acid
TMPTMA = trimethylolpropane trimethacrylate
TMPDE = trimethylolpropane diallyl ether

### Comparative Examples C1-C18 and Examples 1-2: Oil Gel Formulations

Oil gel formulations were prepared having the compositions noted in TABLE 2, by mixing through an overhead mixer first under room temperature at 750 rpm for 15 minutes, followed by another 15 minute mixing at 60° C. at 500 rpm. Heating was applied only after the polymer and silica were fully mixed into the oil.

Rheology profiles were measured at room temperature (25° C.) using a TA Instruments ARES-G2 rheometer with 25 mm parallel plate geometry. Shear sweep method was used with shear rate ranges from 0.01 rad/s to 100 rad/s. Viscosities measured at low shear (0.1 rad/s) and at high shear (100 rad/s) are reported in TABLE 2.

TABLE 2

| | | | | | | | Viscosity | |
|---|---|---|---|---|---|---|---|---|
| | Polymer | | Silica | | Oil | | (Pa · s) | |
| | (wt %) | | (wt %) | | (wt %) | | (0.1 | (100 |
| Ex. | S1 | S2 | M5¹ | TS720² | SSO³ | CCT⁴ | rad/s) | rad/s) |
| C1 | — | — | — | — | 100 | — | 0.08 | 0.05 |
| C2 | — | — | 6.00 | — | 94.0 | — | 41.77 | 0.60 |
| C3 | — | — | — | 6.00 | 94.0 | — | 93.95 | 0.65 |
| C4 | 3.00 | — | 3.00 | 3.00 | 91.0 | — | 196.81 | 154.90 |
| C5 | 5.41 | — | — | 0.59 | 94.0 | — | 1.16 | 0.77 |
| C6 | 5.41 | — | 0.59 | — | 94.0 | — | 2.01 | 0.65 |
| C7 | 4.33 | — | — | 1.67 | 94.0 | — | 6.46 | 0.72 |
| C8 | 3.61 | — | — | 2.39 | 94.0 | — | 22.73 | 0.63 |
| C9* | 5.41 | — | 0.59 | — | 94.0 | — | 1.53 | 0.17 |
| C10* | 4.80 | — | 1.20 | — | 94.0 | — | 1.45 | 0.23 |
| C11 | 5.41 | — | 0.59 | — | — | 94.0 | 22.79 | 3.50 |
| C12 | 4.33 | — | 1.67 | — | — | 94.0 | 17.90 | 1.32 |
| C13 | 3.61 | — | 2.39 | — | — | 94.0 | 31.71 | 1.55 |
| C14 | 6.00 | — | — | — | — | 94.0 | 2.55 | 0.60 |
| C15 | — | — | 6.00 | — | — | 94.0 | — | 2.50 |
| C16 | — | 5.41 | 0.59 | — | — | 94.0 | 5.90 | 1.65 |
| C17 | — | 4.33 | 1.67 | — | — | 94.0 | 85.27 | 1.67 |
| C18 | — | 3.61 | 2.39 | — | — | 94.0 | 1.82 | 0.20 |
| C19 | — | 6.00 | — | — | — | 94.0 | 6.96 | 1.38 |
| 1 | 4.33 | — | 1.67 | — | 94.0 | — | 350.02 | 0.31 |
| 2 | 3.61 | — | 2.39 | — | 94.0 | — | 230.65 | 6.06 |

*in Comparative Examples C9-C10 the silica was added to the polymer during the spray drying process.
¹CAB-O-SIL ® M-5 fumed silica (hydrophilic) available from Cabot Corporation.
²CAB-O-SIL ® TS-720 fumed silica surface treated with polydimethylsiloxane available from Cabot Corporation.
³Sunflower seed oil available from Spectrum Chemical.
⁴Caprylic capric triglyceride oil available from Spectrum Chemical.

We claim:

1. A polymer oil blend comprising:
(a) a cosmetically acceptable long chain hydrophobic ester oil, wherein the cosmetically acceptable long chain hydrophobic ester oil comprises aliphatic C_{>12-24} alkyl triglycerides;
(b) a thickening polymer powder, wherein the thickening polymer powder comprises:
(i) 96 to 99.9 wt %, based on weight of the thickening polymer powder, of structural units of C₄₋₈ alkyl (meth)acrylate monomer;
(ii) 0.1 to 2 wt %, based on weight of the thickening polymer powder, of structural units of (meth)acrylic acid monomer;
(iii) 0 to 2 wt %, based on weight of the thickening polymer powder, of structural units of multi-ethylenically unsaturated crosslinking monomer; and
(c) a silica powder; and
wherein the weight ratio of silica powder/thickening polymer powder is ≥0.25.

2. The polymer oil blend of claim 1, wherein the cosmetically acceptable long chain hydrophobic ester oil is selected from the group consisting of almond oil, andiroba oil, apricot kernel oil, argan oil, avacado oil, babassu oil, borage oil, canola oil, castor oil, coca butter, coconut oil, corn oil, cottonseed oil, crambe oil, cupuacu butter, evening primrose, grape seed oil, hazelnut oil, hybrid safflower oil, illipe butter, Japan wax, jatropha oil, jojoba oil, kokhum butter, linseed oil, mango butter, meadowfoam oil, milk fat, olive oil, ongokea oil, palm kernel oil, palm oil, peanut oil, poppyseed oil, rapeseed oil, rice bran oil, safflower oil, sesame oil, shea butter, soybean oil, sunflower oil, sweet almond oil, tallow, tung oil, walnut oil, wheat germ oil, veronia oil and mixtures thereof.

3. The polymer oil blend of claim 1, wherein the C₄₋₈ alkyl (meth)acrylate monomer is selected from the group consisting of at least one of ethylhexyl (meth)acrylate, n-butyl (meth)acrylate, iso-butyl (meth)acrylate and t-butyl (meth)acrylate.

4. The polymer oil blend of claim 1, wherein 40 to 90 wt % of the structural units of C₄₋₈ alkyl (meth)acrylate monomer are derived from C₄ alkyl (meth)acrylate monomer and wherein 10 to 60 wt % are derived from C₈ (meth)acrylate monomer.

5. The polymer oil blend of claim 4, wherein the C₄ alkyl (meth)acrylate monomer is iso-butyl methacrylate and the C₈ alkyl (meth)acrylate is ethylhexyl methacrylate.

6. The polymer oil blend of claim 1, wherein the thickening polymer comprises 0.05 to 2 wt %, based on weight of the thickening polymer powder, of structural units of multi-ethylenically unsaturated crosslinking monomer.

7. A personal care composition comprising 1 to 100 wt %, based on weight of the personal care composition, of a polymer oil blend of claim 1.

8. The personal care composition of claim 7, further comprising an additive selected from the group consisting of abrasives, absorbents, fragrances, pigments, colorings/colorants, essential oils, skin sensates, astringents, preservatives, anti-caking agents, foam builders, antifoaming agents, antimicrobial agents, antioxidants, binders, biological additives, buffering agents, bulking agents, chelating agents, chemical additives, cosmetic astringents, cosmetic biocides, denaturants, drug astringents, external analgesics, film formers, opacifying agents, pH adjusters, propellants, reducing agents, sequestrants, skin bleaching and lightening agents, skin conditioning agents, skin soothing agents, skin treating agents, vitamins, silicones, fatty alcohols and mixtures thereof.

9. The personal care composition of claim 7, further comprising a cosmetically acceptable surfactant.

10. A method of treating skin or hair, comprising:
providing a personal care composition according to claim 7, and
applying the personal care composition to at least one of skin and hair.
